(19) United States
(12) Patent Application Publication
(10) Pub. No.: US 2007/0173663 A1
(43) Pub. Date: Jul. 26, 2007

(54) Production and use of supported catalysts

(76) Inventors: Atsuyuki Miyaji, Oita (JP); Meiko Saihata, Oita (JP)

Correspondence Address: SUGHRUE MION, PLLC, 2100 PENNSYLVANIA AVENUE, N.W., SUITE 800, WASHINGTON, DC, 20037, US

(21) Appl. No.: 10/591,002

(22) PCT Filed: Dec. 21, 2005
(86) PCT No.: PCT/JP05/24009

§ 371 Date: Aug. 29, 2006

Related U.S. Application Data
(60) Provisional application No. 60/646,645, filed on Jan. 26, 2005.

(30) Foreign Application Priority Data
Jan. 18, 2005 (JP) 2005-010229

(51) Int. Cl. B01J 27/051; B01J 27/047; B01J 27/02; C07C 51/16

(52) U.S. Cl. 562/542; 502/216; 502/219; 502/220

(57) ABSTRACT

Supported catalysts which allow shortening of the production steps for supported catalysts useful for production of lower aliphatic carboxylic acids from lower olefins and oxygen, and which compared to supported catalysts of the prior art, can inhibit generation of carbon dioxide (CO₂) gas by-product during the production of lower aliphatic carboxylic acids. A compound containing at least one element selected from Group 8, 9 and 10 elements of the Periodic Table is loaded on a carrier and then, before reduction treatment of the compound, it is subjected to alkali treatment with an alkaline substance, and a compound containing at least one element selected from gallium, indium, thallium, germanium, tin, lead, phosphorus, arsenic, antimony, bismuth, sulfur, selenium, tellurium and polonium is loaded.

### CROSS-REFERENCE TO RELATED APPLICATION

This application is an application filed under 35 U.S.C. § 111(a) claiming benefit of priority pursuant to 35 U.S.C. § 119(e)(1) of the filing date of the Provisional Application 60/646,645 filed Jan. 26, 2005, pursuant to 35 U.S.C. § 111(b).

### TECHNICAL FIELD

The present invention relates to production and use of supported catalysts. Particularly, the invention relates to production and use of supported catalysts which allow industrially advantageous production of lower aliphatic carboxylic acids such as acetic acid from oxygen and lower olefins such as ethylene in a gas phase.

### BACKGROUND ART

Several processes for single-stage production of acetic acid from ethylene have been proposed because of their many advantages in terms of industrial production and economy. For example, there have been disclosed liquid-phase single-stage oxidation processes using metal ion pair oxidation-reduction catalysts such as palladium-cobalt and palladium-iron (French Patent No. 1448361), processes using catalysts comprising palladium-phosphate or sulfur-containing modifying agents (Japanese Unexamined Patent Publication No. 47-013221, Japanese Unexamined Patent Publication No. 51-029425), and gas-phase single-stage oxidation processes using Group III oxide compounds (Japanese Unexamined Patent Publication No. 46-006763). As acetic acid production processes employing catalysts comprising palladium compounds and heteropolyacids there have also been proposed gas-phase single-stage oxidation processes using catalysts composed of palladium phosphovanadomolybdate (Japanese Unexamined Patent Publication No. 54-57488).

Recently, a catalyst for obtaining acetic acid from ethylene and oxygen has been proposed which is a catalyst comprising metallic palladium and a Periodic Table Group 14, 15 or 16 element supported on a carrier (Japanese Unexamined Patent Publication No. 11-106358).

Such catalysts are prepared by the following steps.

Step 1: A step of loading a palladium-containing compound on a carrier.

Step 2: A step of subjecting the palladium-containing compound to reduction treatment for conversion to metallic palladium.

Step 3: A step of alkali treatment.

Step 4: A step of loading the Group 14, 15 or 16 element.

An egg-shell type catalyst is considered to be most advantageous for this kind of supported catalyst. An egg-shell catalyst is one in which the loading position of the palladium in the carrier is on the outside of the carrier. Because the reaction substrate does not readily diffuse into the interior of the catalyst carrier, the metal component supported inside the carrier has a low probability of contact with the reaction substrate and contributes minimally to the reaction. In an egg-shell catalyst, the metal component is in greater abundance on the carrier surface, and therefore the reaction efficiency is higher than a normal type with the same amount of metal component. An egg-shell type palladium catalyst can be obtained by a known production process comprising an alkali treatment step with sodium metasilicate or the like (Japanese Unexamined Patent Publication No. 7-89896). Also, Japanese Unexamined Patent Publication No. 2000-308830 discloses a process for production of an egg-shell type palladium-supporting catalyst comprising a step of treatment with an alkaline earth metal salt such as barium hydroxide.

The production processes for acetic acid production catalysts disclosed in Japanese Unexamined Patent Publication No. 11-106358 and elsewhere comprise alkali treatment steps in order to preferentially distribute the metal component such as palladium on the carrier surface (egg-shell). Catalysts obtained by the same processes have high catalytic activity but the catalyst modification steps are long and the catalysts can undergo deterioration during the reaction. It has therefore been desired to develop convenient preparation methods for catalysts which maintain high activity while undergoing minimal deterioration.

Carbon dioxide is also generated as a by-product in production processes for obtaining acetic acid by reaction of ethylene and oxygen. For example, Japanese Unexamined Patent Publication No. 7-89896 describes a carbon dioxide selectivity of about 5%. Generation of carbon dioxide basically corresponds to a reduced acetic acid yield. In recent years, inhibiting carbon dioxide production has also become a serious issue in terms of preventing global warming and alleviating the environmental burden. From an industrial standpoint, high equipment investment and high equipment operation and maintenance costs are required for treatment of carbon dioxide by-product. It is therefore desirable to further reduce carbon dioxide by-product during the production of acetic acid.

### DISCLOSURE OF INVENTION

It is an object of the present invention to solve the aforementioned problems of the background art. Specifically, the object of the invention is to shorten the production steps for supported catalysts used in the production of lower aliphatic carboxylic acids such as acetic acid from oxygen and lower olefins such as ethylene, and to provide supported catalysts which allow carbon dioxide gas (CO₂) by-product generation and catalyst deterioration which occur during production of lower aliphatic carboxylic acids to be inhibited compared to the prior art.

As a result of much diligent research on the aforementioned problems, the present inventors completed the present invention based on the discovery of a process for production of supported catalysts (hereinafter also referred to as “supported catalyst preparation process”) characterized by loading on a carrier (a) a compound containing at least one element selected from Group 8, 9 and 10 elements of the Periodic Table (hereinafter referred to as “(a) group compound)”, the compound being subsequently subjected to alkali treatment with an alkaline substance before reduction treatment, and (b) a compound containing at least one element selected from gallium, indium, thallium, germanium, tin, lead, phosphorus, arsenic, antimony, bismuth, sulfur, selenium, tellurium and polonium (hereinafter referred to as “(b) group compound)”.

According to the present invention, “Periodic Table” is the Periodic Table according to the Revised Recommendations for Nomenclature of Inorganic Chemistry (1989) by the IUPAC.

The present invention therefore relates to the following aspects [1] to [16].

[1] A process for production of a supported catalyst comprising the following steps in order.

First Step

A step of impregnating the carrier with a solution containing at least one (a) group compound to obtain an impregnated carrier (A).

Second Step

A step of contacting the impregnated carrier (A) with an alkaline substance and a (b) group compound to obtain an impregnated carrier (B) (where the alkaline substance and the (b) group compound may be contacted simultaneously or separately with the impregnated carrier (A)).

Third Step

A step of contacting the impregnated carrier (B) with a reducing substance to obtain a supported catalyst (C).

[2] A process for production of a supported catalyst according to [1] above, which further comprises a step of loading on the carrier (c) at least one compound selected from heteropolyacids and/or their salts.

[3] A process for production of a supported catalyst according to [1] or [2] above, which further comprises a step of contacting the carrier with (d) at least one compound selected from compounds which contain at least one type of element selected from Group 11 and 12 elements of the Periodic Table and chromium (hereinafter referred to as “(d) group compound”).

[4] A process for production of a supported catalyst according to any one of [1] to [3] above, wherein the (a) group compound is a compound containing at least one type of element selected from ruthenium, osmium, rhodium, iridium, palladium and platinum.

[5] A process for production of a supported catalyst according to any one of [1] to [4] above, wherein the (b) group compound is a compound containing at least one type of element selected from gallium, germanium, tin, lead, bismuth, selenium and tellurium.

[6] A process for production of a supported catalyst according to any one of [1] to [5] above, wherein the polyatom of the (c) heteropolyacid and/or its salt is tungsten and/or molybdenum.

[7] A process for production of a supported catalyst according to any one of [2] to [6] above, wherein the heteroatom of the (c) heteropolyacid and/or its salt is at least one type of element selected from phosphorus, silicon and boron.

[8] A process for production of a supported catalyst according to any one of [2] to [7] above, wherein the (c) heteropolyacid and/or its salt is at least one compound selected from silicotungstic acid, phosphotungstic acid, silicomolybdic acid, phosphomolybdic acid and their salts.

[9] A process for production of a supported catalyst according to any one of [3] to [8] above, wherein the Group 11 or 12 element of the (d) group compound is an element selected from copper, silver, gold and zinc.

[10] A process for production of a supported catalyst according to any one of [1] to [9] above, wherein the supported catalyst is used for a reaction in which a lower aliphatic carboxylic acid is obtained from a lower olefin and oxygen.

[11] A process for production of a supported catalyst according to [10] above, wherein the supported catalyst is used for a reaction in which acetic acid is obtained from ethylene and oxygen.

[12] A supported catalyst obtained by a production process according to any one of [1] to [9] above.

[13] A process for production of a lower aliphatic carboxylic acid, comprising using a supported catalyst according to [12] above for a reaction in which a lower aliphatic carboxylic acid is obtained from a lower olefin and oxygen.

[14] A process for production of a lower aliphatic carboxylic acid according to [13] above, wherein the reaction between the lower olefin and oxygen is carried out in a gas phase.

[15] A process for production of acetic acid, comprising using a supported catalyst according to [12] above is used for reaction to obtain acetic acid from ethylene and oxygen.

[16] A process for production of acetic acid according to [15] above, wherein the reaction between ethylene and oxygen is carried out in a gas phase.

According to the supported catalyst production process of the invention it is possible to shorten the catalyst preparation steps, while the obtained supported catalyst can inhibit carbon dioxide by-product generation and catalyst deterioration in the production of lower aliphatic carboxylic acids from lower olefins and oxygen in a gas phase, thereby providing the advantage of reduced manufacturing costs for lower aliphatic carboxylic acids such as acetic acid.

### BEST MODE FOR CARRYING OUT THE INVENTION

Preferred modes of the invention will now be described in detail.

The supported catalyst obtained by the production process of the invention may be most suitably employed as a catalyst for production of a lower aliphatic carboxylic acid (preferably acetic acid) by reaction of a lower olefin (preferably ethylene) with oxygen in a gas phase.

The process for production of a supported catalyst according to the invention comprises the following steps in order.

First Step

A step of impregnating the carrier with a solution containing at least one (a) group compound to obtain an impregnated carrier (A).

Second Step

A step of contacting the impregnated carrier (A) with an alkaline substance and a (b) group compound to obtain an impregnated carrier (B).

Third Step

A step of contacting the impregnated carrier (B) with a reducing substance to obtain a supported catalyst (C).

In the second step, the alkaline substance and the (b) group compound may be either simultaneously or separately contacted with the impregnated catalyst (A), but simultaneous contact is preferred in order to simplify the step. For separate contact, the alkaline substance is preferably contacted first.

The supported catalyst of the invention more preferably has both a (c) heteropolyacid (and/or its salt) and a (d) group compound supported. So long as the effect of the invention is not impeded, there may also be included a step of loading the (c) compound and the (d) group compound within any of the aforementioned steps or before or after the steps. As explained below, a step of loading the (c) compound is preferably carried out after the third step. Also, a step of contacting at least one (d) group compound with the carrier may be added to the first step, i.e. the (a) group compound and (d) group compound may be loaded on the carrier simultaneously.

According to the background art, an (a) group compound is loaded on a carrier and then contacted with an alkaline substance, subjected to reduction treatment to reduce the (a) group compound to its metallic state (metallic palladium, for example), after which a (b) group compound is loaded on the carrier; however, the present invention is characterized in that the (b) group compound is loaded on the carrier before reduction treatment.

Separate steps may also be included during, or before or after, the aforementioned steps of the invention.

An example of a more preferred supported catalyst production process of the invention will now be explained in detail.

First Step

A step of impregnating the carrier with a solution containing an (a) group compound (for example, a compound containing Pd) and a (d) group compound (for example, a compound containing Au or Zn) to obtain an impregnated carrier (A).

Second Step

A step of contacting the impregnated carrier (A) with an alkaline substance and a (b) group compound (for example, a compound containing Te) to obtain an impregnated carrier (B).

Third Step

A step of contacting the impregnated carrier (B) with a reducing substance (for example, hydrazine) for reduction of the (a) group compound to obtain a supported catalyst (C).

Fourth Step

A step of loading a (c) heteropolyacid (for example, silicotungstic acid) or its salt on the supported catalyst (C).

<First Step>

<Carrier>

There are no particular restrictions on the carrier used for production of a supported catalyst of the invention, but it is preferably a porous substance which is commonly used for carriers. Specifically, there may be mentioned silica, silica-alumina, diatomaceous earth, montmorillonite, titania or the like. Silica is especially preferred.

The form of the carrier is not particularly restricted. Specifically, there may be mentioned a powder form, globule form or pellet form. The optimum form may be selected to conform to the reaction system and reaction vessel used.

The sizes of the carrier particles are also not particularly restricted. For a globular carrier used in a fixed-bed tubular reactor, the particle diameters are preferably 1-10 mm, and more preferably 2-8 mm. When a tubular reactor is packed with a supported catalyst for reaction, a particle diameter of smaller than 1 mm can produce significant pressure loss of the gas flow, potentially preventing effective circulation of gas. A particle diameter of larger than 10 mm, on the other hand, can prevent diffusion of the reaction gas into the catalyst interior, potentially preventing the catalytic reaction from proceeding efficiently. The pore structure of the carrier is preferably one with pore sizes of 1-1000 nm, and more preferably 3-200 nm. The area to weight ratio of the carrier as measured by the BET method is preferably 30-700 m²/g and more preferably 50-300 m²/g. The bulk density of the carrier is preferably 50-1000 g/l and more preferably 300-500 g/l.

<(a) Group Compound>

The (a) group compound is a compound containing at least one element selected from Group 8, 9 and 10 elements of the Periodic Table. Group 8, 9 and 10 elements of the Periodic Table include iron, ruthenium, osmium, cobalt, rhodium, iridium, nickel, palladium and platinum, but palladium, platinum and nickel are preferred, with palladium being particularly preferred.

The (a) group compound may be in any physical state. It may be a compound containing a Group 8, 9 or 10 element or it may be the element itself. In other words, the element may be in ionic form in the compound, or it may be in the metallic state with a valence of 0.

As (a) group compounds there may be mentioned metallic palladium or metallic platinum, metallic nickel, halides such as palladium chloride, platinic chloride or nickel chloride, organic acid salts such as palladium acetate or platinum acetate, nitric acid salts such as palladium nitrate, platinum nitrate or nickel nitrate, or palladium oxide, nickel oxide, sodium tetrachloropalladate or potassium tetrachloropalladate, as well as chelates having organic compounds such as acetylacetonate, nitrile or ammonium as ligands. Particularly preferred are sodium tetrachloropalladate, hexachloroplatinic acid, potassium tetrachloropalladate and palladium nitrate. These (a) group compounds may be used alone, or different compounds may be used in combination.

The supported form of the (a) group compound on the carrier is preferably an “egg-shell type”. For an egg-shell type supported catalyst, the method of loading the (a) group compound on the carrier is not particularly restricted so long as it results in an egg-shell type supported catalyst. An egg-shell type catalyst is one form of distribution of an active component (for example, metallic palladium) on carrier particles or in a compact, wherein virtually all of the active component is present on the outer surface of the carrier particles or compact. Specifically, there may be mentioned a method of direct loading or indirect loading of the starting compound on the surface layer, after dissolution in an appropriate solvent such as water or acetone, an inorganic acid or organic acid such as hydrochloric acid, nitric acid or acetic acid, or a solution thereof. As direct loading methods there may be mentioned impregnation and spraying. As an indirect loading method there may be mentioned a method of first loading the (a) group compound on the carrier (first step), and causing the (a) group compound in the interior to migrate to the surface by alkali treatment (second step), followed by reduction (third step), as in the process described above.

Loading of the (a) group compound on the carrier may be accomplished by preparing a uniform solution containing at least one (a) group compound and impregnating the carrier with an appropriate amount of the solution. More specifically, the (a) group compound may be dissolved in an appropriate solvent such as water or acetone, or in an inorganic or organic acid such as hydrochloric acid, nitric acid or acetic acid, to form a uniform solution, and then the carrier impregnated with this solution to obtain an impregnated carrier (A). The impregnation may be followed by drying, but it is preferred to proceed to the second step without a drying step because this will simplify the process.

<Second Step>

<Alkaline Substance>

The alkaline substance used for the second step may be supplied as a solution or as a gas. Preferably it is a solution in water or alcohol. The solute may be a hydroxide or silicate of an alkali metal, and is preferably potassium hydroxide, sodium hydroxide, sodium metasilicate and/or barium hydroxide. All or a portion of the palladium compound may be converted to an oxide or hydroxide in this step.

<(b) Group Compound>

The (b) group compound is a compound containing at least one element selected from gallium, indium, thallium, germanium, tin, lead, phosphorus, arsenic, antimony, bismuth, sulfur, selenium, tellurium and polonium. The “compound containing at least one element” may be the element itself (a metal) or a chloride, nitrate, acetate, phosphate, sulfate or oxide of the element, or even a chelate comprising an organic compound such as acetylacetonate or nitrile as the ligand.

As elements in the (b) group compound there are preferred gallium, germanium, tin, lead, arsenic, antimony, bismuth, selenium, tellurium and polonium, among which tellurium is particularly preferred.

As specific examples of (b) group compounds there may be mentioned sodium tellurite (Na₂TeO₃), potassium tellurite (K₂TeO₃), sodium tellurate (Na₂TeO₄) and potassium tellurate (K₂TeO₄).

In the second step, the impregnated carrier (B) may be obtained by contacting a solution of the alkaline substance and the (b) group compound with the impregnated carrier (A). Alternatively, the impregnated carrier (A) may be contacted with a solution of the alkaline substance and then with a solution of the (b) group compound, or this order may be reversed. The solvent used is preferably water and/or an alcohol, and more preferably water.

<Third Step>

In the third step, a reducing substance is contacted with the impregnated carrier (B) for reduction treatment.

The reduction treatment is preferably carried out on the carrier supporting the (a) group compound and the (b) group compound. By this procedure, the (a) group compound can interact with the (b) group compound while in an ion state.

The reduction treatment may also be carried out after first loading the (c) heteropolyacid and/or its salt on the impregnated carrier (B). That is, the third and fourth steps may be switched. The following is an example.

First step: A step in which the carrier is impregnated with a solution containing an (a) group compound to obtain an impregnated carrier (A).

Second step: A step in which the impregnated carrier (A) is contacted with a solution containing an alkaline substance and a (b) group compound to obtain an impregnated carrier (B).

Fourth step: A step in which the impregnated carrier (B) is impregnated with a solution containing a (c) heteropolyacid to obtain a supported catalyst (D).

Third step: A step in which the supported catalyst (D) is subjected to reduction treatment.

The reduction treatment may be carried out after isolating the impregnated carrier (A) or (B), or it may be carried out following the loading procedure. Alternatively, instead of reducing all of the supported (a) group compound, only a portion thereof may be reduced.

Reducing substances include hydrazine, hydrogen, ethylene, carbon monoxide and the like. These substances may be contacted with the impregnated carrier (B) or the supported catalyst (C) in a liquid phase or a gas phase to reduce the (a) group compound.

When the reduction treatment is carried out by a liquid phase method, there is no particular restriction on the temperature, but it is preferred for the impregnated carrier (B) or the supported catalyst (C) to be at a temperature of about 10-200° C. The temperature is more preferably 20-100° C.

When the reduction treatment is carried out by a gas phase method, the temperature is not particularly restricted but the impregnated carrier (B) or the supported catalyst (C) is preferably heated to around 30-350° C. The temperature is more preferably 100-300° C. When the heteropolyacid is loaded first, the reaction is preferably not carried out at above 350° C. because this may cause decomposition of the heteropolyacid.

A treatment pressure of 0.0-3.0 MPaG (gauge pressure) for reduction treatment by a gas phase method is advantageous in practical terms from the standpoint of the equipment, but there is no particular restriction. The pressure range is more preferably 0.1-1.5 MPaG (gauge pressure).

For flow of a gaseous reductive substance, the reductive substance may be at any concentration, and if necessary nitrogen, carbon dioxide or a rare gas may be used as a diluent. The reduction may also be carried out using ethylene, hydrogen or the like in the presence of vaporized water. Alternatively, the catalyst may be filled into the reactor before the reduction treatment and reduced with ethylene, and then oxygen introduced for production of acetic acid from the ethylene and oxygen.

A mixed gas containing the gaseous reducing substance is preferably contacted with the catalyst under standard conditions at a space velocity (SV) of 10-15,000 hr⁻¹ and especially 100-8000 hr⁻¹.

The treatment system is not particularly restricted, but for practical advantages it is preferred to employ a fixed-bed having the catalyst packed in a corrosion-resistant reaction tube.

<Fourth Step>

<(c) Heteropolyacid>

The (c) heteropolyacid used for the invention is preferably a heteropolyacid comprising tungsten or molybdenum as the polyacid. As hetero acids there may be mentioned phosphorus, silicon, boron, aluminum, germanium, titanium, zirconium, cerium, cobalt and chromium, but there is no restriction to these. Phosphorus, silicon and boron are preferred.

As specific examples of heteropolyacids there may be mentioned silicotungstic acid, phosphotungstic acid, silicomolybdic acid, phosphomolybdic acid and borotungstic acid. Preferred are silicotungstic acid, phosphotungstic acid, silicomolybdic acid and phosphomolybdic acid shown below. The structure of the polyacid is not particularly restricted, but preferably the heteropolyacid has a Keggin-type structure.

Silicotungstic acid: H₄SiW₁₂O₄₀.nH₂O

Phosphotungstic acid: H₃PW₁₂O₄₀.nH₂O

Silicomolybdic acid: H₄SiMo₁₂O₄₀.nH₂O

Phosphomolybdic acid: H₄PMo₁₂O₄₀.nH₂O

(wherein n represents 0 or an integer of 1-40)

A salt of the (c) heteropolyacid used for the invention is a metal salt or onium salt wherein two or more different inorganic oxyacids are condensed to produce an acid and all or a portion of the hydrogen atoms are replaced. The metals replacing the hydrogen atoms of the heteropolyacid are preferably at least one type of element selected from the group consisting of Group 1, Group 2, Group 11 and Group 13 elements of the Periodic Table, and examples of onium salts of heteropolyacids include their ammonium salts. Particularly preferred among such heteropolyacid salts are salts of such metals as lithium, sodium, potassium, cesium, rubidium, calcium, magnesium, barium, copper, gold, silver and gallium.

As preferred heteropolyacid salts from the standpoint of catalyst performance there may be mentioned lithium salt of phosphotungstic acid, sodium salt of phosphotungstic acid, copper salt of phosphotungstic acid, lithium salt of silicotungstic acid, sodium salt of silicotungstic acid and copper salt of silicotungstic acid.

The (c) heteropolyacid and/or its salt may be of a single type, or a combination of more than one type. The method for loading the heteropolyacid and/or its salt on the carrier may be an impregnation method, spray method, or other means. The solvent used for impregnation is preferably one which dissolves the (c) heteropolyacid and its salts, and water, organic solvents or mixtures thereof may be used. Preferred solvents are water, alcohols and ethers.

The loading step for the (c) heteropolyacid and/or its salt onto the carrier preferably follows the third step (reduction treatment), but it may optionally be carried out before the third step, as mentioned above. Alternatively, it may also be included within the first step. That is, the (a) group compound and the (c) heteropolyacid and/or its salt may be simultaneously loaded in the first step. It may instead be loaded separately from the (a) group compound either before or immediately after the first step.

The method for simultaneously loading the (a) group compound and the (c) heteropolyacid and/or its salt in the first step may be a method in which a uniform solution of the (a) group compound and the (c) heteropolyacid and/or its salt is used for simultaneous loading onto the carrier. More specifically, the (a) group compound and the (c) heteropolyacid and/or its salt is dissolved in an appropriate solvent such as water or acetone or an inorganic or organic acid such as hydrochloric acid, nitric acid or acetic acid to form a uniform solution, which is then impregnated into the carrier and dried. Alternatively, a heteropolyacid metal salt prepared from the (a) group compound and the (c) heteropolyacid and/or its salt may be obtained first, and then dissolved in an appropriate solvent for loading. Preferred heteropolyacids for the heteropolyacid acid metal salt include phosphotungstic acid, silicotungstic acid, phosphomolybdic acid and silicomolybdic acid, and palladium may be mentioned as a metal.

As a method of loading the (c) heteropolyacid and/or its salt separately from the (a) group compound either immediately before or after the first step, there may be mentioned a method in which an aqueous solution of (a) or (c) is prepared and the carrier is impregnated with the (a) group compound or the (c) compound for loading of the (a) group compound or (c) compound. The order of loading the (a) group compound or (c) compound may be as desired. More specifically, the (a) group compound or the (c) compound is dissolved in an appropriate solvent such as water or acetone or an inorganic or organic acid such as hydrochloric acid, nitric acid or acetic acid to form a uniform solution of each, and then the carrier is impregnated with the uniform solution of the (a) group compound or (c) compound and then dried, after which it is then impregnated with the uniform solution of either the (c) compound or the (a) group compound and dried.

<(d) Group Compound>

The stage for the step of loading the (d) group compound is not particularly restricted. For example, it may be loaded simultaneously with the (a) group compound and the (b) group compound, or with the (c) heteropolyacid and/or its salt, or it may be loaded independently. A more preferred loading method is a method wherein it is loaded simultaneously with the (a) group compound.

The (d) group compound is a compound which contains at least one element selected from among Group 11 and 12 elements of the Periodic Table and chromium. The included element is preferably Cr, Au or Zn, with Au and Zn being more preferred.

There are no particular restrictions on (d) group compounds so long as they are compounds comprising at least one element selected from among Group 11 and 12 elements of the Periodic Table or chromium. As examples there may be mentioned the elements themselves (metallic state), or chlorides, nitrates, acetates, phosphates, sulfates, oxides and the like containing the elements, as well as chelates comprising organic compounds such as acetylacetonato or nitriles as ligands. Most preferably there may be mentioned zinc chloride and auric chloride.

<Catalyst for Production of Lower Aliphatic Carboxylic Acid>

There are no particular restrictions on the composition of the (a) group compound, (b) group compound, (c) heteropolyacid (and/or its salt) and (d) group compound in the catalyst having (a), (b), (c) and (d) held on the carrier in the catalyst for production of the lower aliphatic carboxylic acid obtained by the supported catalyst production process of the invention. Preferably, the weight percentages in the entire supported catalyst are (a):(b):(c):(d)=0.5-5 wt %:0.05-3.0 wt %:5-50 wt %:0.05-3.0 wt %, and most preferably (a):(b):(c):(d)=1.0-2.5 wt %:0.08-1.0 wt %:10-40 wt %:0.08-1.0 wt %. When each compound group consists of a plurality of compounds, their respective totals are used for the compositional ratios of each component. The other components of the carrier are also present in addition to components (a), (b), (c) and (d).

Drying of the catalyst after loading solutions of one or more compounds of (a), (b), (c), (d) or their salts may be accomplished by any method. For example, there may be mentioned a method of vacuum treatment at low temperature, and a method of removal of the solvent by heat treatment in a hot air drier.

The loading amounts and compositional ratios of the metal element and heteropolyacid in the supported catalyst for production of a lower aliphatic carboxylic acid, which is produced according to the invention, can be measured to high precision with chemical analysis such as by a high-frequency inductively coupled plasma (ICP) device, fluorescent X-ray analysis (XRF) or atomic absorption analysis.

As an example of a measuring method, a prescribed amount of catalyst is pulverized with a mortar to produce a uniform powder, and then the catalyst powder is added to an acid such as hydrofluoric acid or aqua regia and stirred while heating for dissolution to form a homogeneous solution. The solution is then diluted to a suitable concentration with purified water to prepare an analysis solution. The solution may be quantitatively analyzed by ICP.

The production steps for a lower aliphatic carboxylic acid using a catalyst obtained according to the invention will now be explained, using for simplicity an example where a supported catalyst of the invention is used to obtain acetic acid by gas phase reaction of ethylene and oxygen in a fixed-bed circulating reactor.

In a process for production of acetic acid according to the invention, there is no particular restriction on the reaction temperature for the reaction between ethylene and oxygen to produce acetic acid. The temperature is preferably 100-300° C. and more preferably 120-250° C. A reaction pressure of 0.0-3.0 MPaG (gauge pressure) is advantageous in practical terms from the standpoint of the equipment, but there is no particular restriction. The pressure range is more preferably in the range of 0.1-1.5 MPaG (gauge pressure).

The gas supplied to the reaction system comprises ethylene and oxygen, and if necessary a diluent such as nitrogen, carbon dioxide, or a rare gas may also be used.

There are preferably supplied to the reaction system ethylene in an amount for a proportion of 5-80 vol % and preferably 8-50 vol % and oxygen in an amount for a proportion of 1-15 vol % and preferably 3-12 vol %, based on the total amount of supplied gas.

Adding water to the reaction system has a notable effect of improving the acetic acid productivity and selectivity, and maintaining the activity of the catalyst. Water vapor is preferably included in the reaction gas in a range of 1-50 vol %, but more preferably 5-40 vol %.

A high purity ethylene starting material is preferably used for the reaction system, but there is no problem with contamination by lower saturated hydrocarbons such as methane, ethane or propane. The oxygen may be supplied in a form diluted with an inert gas such as nitrogen or carbon dioxide gas, such as air for example, but when the reaction gas is circulated, it is generally advantageous to have a high concentration of oxygen at preferably 99% or greater.

The reaction mixture gas is preferably circulated into the catalyst at SV=10-15,000 h⁻¹ and especially 300-8000 h⁻¹ under standard conditions.

The mode of reaction is not particularly restricted, and a publicly known reaction system such as a fixed bed or fluidized bed may be employed. Preferred from a practical standpoint is a fixed bed having the catalyst packed into a corrosion-resistant reaction tube.

The present invention will now be explained in greater detail by examples, with the understanding that the invention is not limited only to these examples.

<Pretreatment of Carriers>

All of the carriers used in the examples were pretreated by drying in air at 110° C. for 4 hours.

<Water>

All of the water used in the examples was deionized water.

<Carriers>

The carriers used in the examples were all silica carriers [BET area to weight ratio: 148 m²/g, bulk density: 405 g/l, 5 mmφ, product of Shangai Haiynan Chemical Industry Science & Technology Corp. Ltd.]

<Starting Compounds>

Aqueous hydrochloric acid solution of sodium palladium chloride [Na₂PdCl₄] and aqueous nitric acid solution of palladium nitrate [Pd(NO₃)₂] (N. E. Chemcat Co., Ltd.)

Silicotungstic acid.26H₂O [H₄SiW₁₂O₄₀.26H₂O] (Nippon Inorganic Colour & Chemical Co., Ltd.)

Zinc chloride [ZnCl₂] (Wako Pure Chemical Industries Co., Ltd.)

Auric chloride [HAuCl₄.4H₂O] (Wako Pure Chemical Industries Co., Ltd.)

Sodium metasilicate nonahydrate [Na₂SiO₃.9H₂O] (Wako Pure Chemical Industries Co., Ltd.)

Sodium tellurite [Na₂TeO₃] (Wako Pure Chemical Industries Co., Ltd.)

Hydrazine monohydrate [N₂H₄.H₂O] (Wako Pure Chemical Industries Co., Ltd.)

Silicotungstic acid.26H₂O (Nippon Inorganic Colour & Chemical Co., Ltd.)

Phosphomolybdic acid.30H₂O (Nippon Inorganic Colour & Chemical Co., Ltd.)

### EXAMPLE 1

After mixing 2.47 g of sodium palladium chloride prepared to 20.24 wt %, 1.3 g of aqueous zinc chloride prepared to 4.5 wt % and 4.0 g of aqueous auric chloride prepared to 10 wt %, the mixture was increased in volume with ion-exchanged water to prepare a 20 ml aqueous solution (A-1 solution). This A-1 solution was impregnated into a silica carrier (50 g) to absorption of the total amount. Next, 8.0 g of sodium metasilicate nonahydrate and 200 mg of sodium tellurite were dissolved in 90 ml of water to obtain an aqueous solution (B-1), which was impregnated into a silica carrier and allowed to stand at room temperature for 20 hours. After further adding 6.5 g of hydrazine monohydrate and gently stirring the mixture, it was allowed to stand at room temperature for 4 hours. The catalyst was filtered out and then transferred to a stop cock-equipped glass column, and purified water was flowed through for 40 hours for washing. This was followed by drying under an air stream at 110° C. for 4 hours to obtain an impregnated carrier.

Also, 20.7 g of silicotungstic acid.26H₂O was prepared as a homogeneous solution and increased in volume to 45 ml (C-1 solution). The previously prepared impregnated carrier was then impregnated with this C-1 solution to absorption of the total amount. This was followed by drying under an air stream at 110° C. for 4 hours to obtain an acetic acid production catalyst 1.

### COMPARATIVE EXAMPLE 1

After mixing 2.47 g of sodium palladium chloride prepared to 20.24 wt %, 1.3 g of aqueous zinc chloride prepared to 4.5 wt % and 4.0 g of aqueous auric chloride prepared to 10 wt %, the mixture was increased in volume with ion-exchanged water to prepare a 20 ml aqueous solution (A-2 solution). This A-2 solution was impregnated into a silica carrier (50 g) to absorption of the total amount. Next, 8.0 g of sodium metasilicate nonahydrate was dissolved in 90 ml of water to obtain an aqueous solution (B-2), which was impregnated into a silica carrier and allowed to stand at room temperature for 20 hours. After further adding 6.5 g of hydrazine monohydrate and gently stirring the mixture, it was allowed to stand at room temperature for 4 hours. The catalyst was filtered out and then transferred to a stop cock-equipped glass column, and purified water was flowed through for 40 hours for washing. This was followed by drying under an air stream at 110° C. for 4 hours to obtain an impregnated carrier (A-2).

An aqueous solution (C-2) was also prepared by dissolving 200 mg of sodium tellurite in 45 ml of water. The obtained aqueous solution (C-2) was impregnated into the impregnated carrier (A-2), and then the catalyst was filtered out and transferred to a stop cock-equipped glass column, after which purified water was circulated through for 20 hours for washing. This was followed by drying under an air stream at 110° C. for 4 hours to obtain an impregnated carrier (B-2).

Also, 20.7 g of silicotungstic acid.26H₂O was prepared as a homogeneous solution and increased in volume to 45 ml (D-2 solution). The previously prepared impregnated carrier (B-2) was then impregnated with this D-2 solution to absorption of the total amount. This was followed by drying under an air stream at 110° C. for 4 hours to obtain an acetic acid production catalyst 2.

### EXAMPLE 2

A catalyst was prepared in the same manner as Example 1 except that instead of the C-1 solution used in Example 1, 20.7 g of silicotungstic acid.26H₂O and 0.13 g of phosphomolybdic acid.30H₂O (total: 20.83 g) were homogeneously dissolved and increased in volume to 45 ml to form a solution (C-3 solution), in order to obtain an acetic acid production catalyst 3.

### COMPARATIVE EXAMPLE 2

A catalyst was prepared in the same manner as Comparative Example 1 except that instead of the D-2 solution used in Comparative Example 1, 20.7 g of silicotungstic acid.26H₂O and 0.13 g of phosphomolybdic acid.30H₂O (total: 20.83 g) were homogeneously dissolved and increased in volume to 45 ml to form a solution (D-3 solution), in order to obtain an acetic acid production catalyst 4.

### EXAMPLE 3 AND COMPARATIVE EXAMPLE 3

A 5 ml portion each of the acetic acid production catalyst 1 obtained in Example 1 and the acetic acid production catalyst 2 obtained in Comparative Example 1 was homogeneously diluted with 11 ml of silica and then packed into a SUS316L reaction tube (inner diameter: 25 mm), and a gas mixture having an ethylene:oxygen:water:nitrogen volume ratio of 10:6:25:59 was introduced at a space velocity of 9000 h⁻¹, with a catalyst layer reaction peak temperature of 200° C. and a reaction pressure of 0.8 MPaG (gauge pressure), for reaction to obtain acetic acid from ethylene and oxygen.

As the analysis method for the reaction, the total amount of exiting gas which had passed through the catalyst-packed layer was cooled, and the total amount of the collected reaction solution was recovered and subjected to gas chromatography. The total amount of the non-condensed gas which had flowed out during the sampling period was measured and a portion thereof was removed and its composition analyzed by gas chromatography. The generated gas was cooled, and the cooled condensate liquid and the gas components were both analyzed by gas chromatography (GC-14B by Shimadzu Laboratories, FID detector: TC-WAX Capillary Column (30 m length, 0.25 mm inner diameter, 0.25 μm film thickness)).

The catalyst activity was calculated as the mass of acetic acid produced per catalyst volume (liter) per unit time (space-time yield: STY, units: g/hL_{cat}). The carbon dioxide selectivity was determined by the following formula.
CO₂ selectivity (based on carbon)(%)=(CO₂ generation)/(product yield)×100

The acetic acid STY at the start of the reaction and the carbon dioxide selectivity are shown in Table 1. Table 1 shows that the catalyst of Example 3 is a superior catalyst to that of Comparative Example 3 in terms of inhibiting carbon dioxide selectivity.

TABLE 1

| | | | CO₂ |
|---|---|---|---|
| | | Acetic acid | selectivity |
| | Catalyst | STY (g h⁻¹ L⁻¹) | (%) |
| Example 3 | 1 | 548.9 | 3.5 |
| Comp. Example 3 | 2 | 548.8 | 6.0 |

### EXAMPLE 4 AND COMPARATIVE EXAMPLES 4 AND 5

After uniformly diluting 50 ml each of the acetic acid production catalysts 3, 4 and 2 obtained in Example 2 and Comparative Examples 1 and 2 with 150 ml of silica, it was packed into a SUS316L reaction tube (inner diameter: 27 mm), and a gas mixture having an ethylene:oxygen:water:nitrogen volume ratio of 10:6:25:59 was introduced at a space velocity of 4000 h⁻¹, with a catalyst layer reaction peak temperature of 210° C. and a reaction pressure of 0.68 MPaG (gauge pressure), for reaction to obtain acetic acid from ethylene and oxygen.

The acetic acid STY and amount of reduction between start of the reaction and 1000 hours after start of the reaction were determined by the same analysis method as in Example 4. The acetic acid STY reduction per 1000 hours was determined by the following formula.
Acetic acid STY reduction per 1000 hr={(STY_{y})−(STYₓ)}1000/(y−x)

x: Reaction time (start of reaction)

y: Reaction time (approximately 1000 hr)

Table 2 shows that the catalyst of Example 4 may be considered a superior catalyst to those of Comparative Examples 4 and 5 in terms of deterioration of catalyst performance.

TABLE 2

| | | | | Acetic acid |
|---|---|---|---|---|
| | | | | STY |
| | | | | reduction |
| | | Reaction | | per 1000 h |
| | Catalyst | time (h) | STY (g h⁻¹ L⁻¹) | (g h⁻¹ L⁻¹) |
| Example 4 | 3 | 21 | 326.9 | 74.0 |
| | | 1006 | 254.1 | |
| Comp. Example 4 | 4 | 20 | 341.2 | 87.4 |
| | | 1029 | 253.1 | |
| Comp. Example 5 | 2 | 17 | 346.6 | 105.4 |
| | | 1001 | 242.9 | |

### INDUSTRIAL APPLICABILITY

The present invention is a production process for a carrier-supported catalyst containing an (a) group compound and a (b) group compound, characterized in that loading of the (b) group compound is carried out at least before the reduction step, whereby it is possible to shorten the catalyst preparation step compared to prior art production processes. Consequently, the process is not only economically advantageous but is also of high industrial value since use of the obtained acetic acid production catalyst results in inhibited generation of carbon dioxide by-product.

1. A process for production of a supported catalyst comprising the following steps in order:
First step
A step of impregnating the carrier with a solution containing (a) at least one compound which comprises at least one element selected from Group 8, 9 and 10 elements of the Periodic Table (hereinafter referred to as “(a) group compound)”, to obtain an impregnated carrier (A)
Second step
A step of contacting the impregnated carrier (A) with an alkaline substance and (b) a compound containing at least one element selected from among gallium, indium, thallium, germanium, tin, lead, phosphorus, arsenic, antimony, bismuth, sulfur, selenium, tellurium and polonium (hereinafter referred to as “(b) group compound)” to obtain an impregnated carrier (B) (where the alkaline substance and the (b) group compound may be contacted simultaneously or separately with the impregnated carrier (A))
Third step
A step of contacting the impregnated carrier (B) with a reducing substance to obtain a supported catalyst (C).

2. The process for production of a supported catalyst according to claim 1, which further comprises a step of loading on the carrier (c) at least one compound selected from heteropolyacids and/or their salts.

3. The process for production of a supported catalyst according to claim 1, which further comprises a step of contacting the carrier with (d) at least one compound selected from compounds which contain at least one element selected from Group 11 and 12 elements of the Periodic Table and chromium (hereinafter referred to as “(d) group compound”).

4. The process for production of a supported catalyst according to claim 1, wherein the (a) group compound is a compound containing at least one element selected from ruthenium, osmium, rhodium, iridium, palladium and platinum.

5. The process for production of a supported catalyst according to claim 1, wherein the (b) group compound is a compound containing at least one element selected from gallium, germanium, tin, lead, bismuth, selenium and tellurium.

6. The process for production of a supported catalyst according to claim 2, wherein the polyatom of the (c) heteropolyacid and/or its salt is tungsten and/or molybdenum.

7. The process for production of a supported catalyst according to claim 2, wherein the heteroatom of the (c) heteropolyacid and/or its salt is at least one element selected from phosphorus, silicon and boron.

8. The process for production of a supported catalyst according to claim 2, wherein the (c) heteropolyacid and/or its salt is at least one compound selected from silicotungstic acid, phosphotungstic acid, silicomolybdic acid, phosphomolybdic acid and their salts.

9. The process for production of a supported catalyst according to claim 3, wherein the Group 11 or 12 element of the (d) group compound is an element selected from copper, silver, gold and zinc.

10. The process for production of a supported catalyst according to claim 1, wherein the supported catalyst is used for a reaction in which a lower aliphatic carboxylic acid is obtained from a lower olefin and oxygen.

11. The process for production of a supported catalyst according to claim 10, wherein the supported catalyst is used for a reaction in which acetic acid is obtained from ethylene and oxygen.

12. A supported catalyst obtained by a production process according to claim 1.

13. A process for production of a lower aliphatic carboxylic acid, comprising using a supported catalyst according to claim 12 for a reaction in which a lower aliphatic carboxylic acid is obtained from a lower olefin and oxygen.

14. The process for production of a lower aliphatic carboxylic acid according to claim 13, wherein the reaction between the lower olefin and oxygen is carried out in a gas phase.

15. A process for production of acetic acid, comprising using a supported catalyst according to claim 12 for reaction to obtain acetic acid from ethylene and oxygen.

16. The process for production of acetic acid according to claim 15, wherein the reaction between ethylene and oxygen is carried out in a gas phase.

17. The process for production of a supported catalyst according to claim 2, which further comprises a step of contacting the carrier with (d) at least one compound selected from compounds which contain at least one element selected from Group 11 and 12 elements of the Periodic Table and chromium (hereinafter referred to as “(d) group compound”).
